(19) United States
(12) Patent Application Publication
(10) Pub. No.: US 2008/0033183 A1
(43) Pub. Date: Feb. 7, 2008

(54) PROCESS FOR PREPARING PYRANOINDAZOLE SEROTONERGIC RECEPTOR AGONISTS

(75) Inventor: Raymond E. CONROW, Crowley, TX (US)

Correspondence Address: ALCON, IP LEGAL, TB4-8, 6201 SOUTH FREEWAY, FORT WORTH, TX, 76134, US

(73) Assignee: ALCON MANUFACTURING, LTD., Fort Worth, TX (US)

(21) Appl. No.: 11/832,531

(22) Filed: Aug. 1, 2007

Related U.S. Application Data
(60) Provisional application No. 60/821,104, filed on Aug. 1, 2006.

(51) Int. Cl. C07D 491/02

(52) U.S. Cl. 548/359.5

(57) ABSTRACT

Described are methods of making pyranoindazoles comprising reacting with a reducing agent a protected halohydrin comprising a secondary carbamate to form a pyranoindazole. In preferred embodiments the secondary carbamate is a benzyl carbamate. Also preferred are embodiments wherein the reacting is preceded by reacting a protected halohydrin with a first organometallic compound. The pyranoindazoles thus formed by the described methods are preferably pharmaceutically active products.

### CROSS-REFERENCE TO RELATED APPLICATION

This application claims priority under 35 U.S.C. §119 to U.S. Provisional Patent Application No. 60/821,104, filed Aug. 1, 2006, the entire contents of which are incorporated herein by reference.

### TECHNICAL FIELD OF THE INVENTION

The present invention is related generally to processes for preparing pyranoindazole serotonergic receptor agonists and more specifically to processes for preparing pyranoindazole 5-HT receptor agonists useful for the treatment of glaucoma.

### BACKGROUND OF THE INVENTION

Serotonergic receptor agonists are being investigated as compounds useful for treating a variety of disease states, including the ocular disease glaucoma. The disease state referred to as glaucoma is characterized by a permanent loss of visual function due to irreversible damage to the optic nerve. The several morphologically or functionally distinct types of glaucoma are typically characterized by elevated intraocular pressure (IOP), which is considered to be causally related to the pathological course of the disease. If glaucoma or ocular hypertension is detected early and treated promptly with medications that effectively reduce elevated intraocular pressure, loss of visual function or its progressive deterioration can generally be ameliorated. There is, therefore, a need for therapeutic agents that modulate IOP.

5-HT serotonergic receptor agonists have been disclosed as having utility as agents for treating glaucoma and elevated IOP in U.S. Pat. No. 6,696,476 to Chen et al., issued Feb. 24, 2004, the entire contents of which are herein incorporated by reference. U.S. Pat. No. 6,696,476 teaches in part the synthesis of pyranoindazole compounds via pyran ring formation as shown in Scheme 1, compounds 4 to 8 and explained in further detail in Example 2 of that disclosure. U.S. Pat. No. 6,998,489 to Conrow et al., issued Feb. 14, 2006, teaches the synthesis of indazole compounds, the entire contents of which are herein incorporated by reference. It is an object of the present invention to provide additional intermediates and processes for the synthesis of such or similar pyranoindazole compounds that may be useful as serotonergic receptor agonists and/or glaucoma treatment agents. Other objects will be evident from the ensuing description and claims.

### SUMMARY OF THE INVENTION

The present invention is directed to processes for the synthesis of pyranoindazoles, particularly pyranoindazoles that are serotonergic receptor agonists. Embodiments of the present invention provide efficient and simplified methods for the synthesis of such indazole compounds.

In one embodiment of the present invention, a pyranoindazole compound may be formed by reductive cyclization of a dihalide comprising a secondary carbamate, which is then converted to a primary amino group via hydrogenolysis. In a preferred embodiment, the pyranoindazole compound thus formed is (R)-1-((S)-2-aminopropyl)-1,7,8,9-tetrahydropyrano[2,3-g]indazol-8-ol (1):

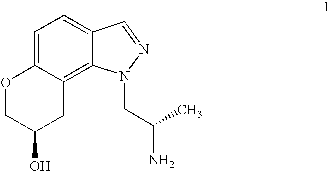

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures.

### DETAILED DESCRIPTION OF THE INVENTION

The present invention provides processes for preparing pyranoindazole 5-HT serotonergic receptor agonists by reductive cyclization. Such agonists may be particularly useful for treating the eye disease glaucoma.

In the procedure described in U.S. Pat. No. 6,696,476, the use of a siloxy group in the sidechain linked to N-1 requires numerous steps after the pyran ring formation to convert it to a primary amino group found in desired serotonergic agonists such as (1). The extended cyclization sequence disclosed herein provides a shorter, more efficient synthesis of the desired serotonergic agonists.

In certain embodiments of the present invention, the cyclization sequence described in Example 2, Steps D-G and Scheme 1, compounds 4-8 of U.S. Pat. No. 6,696,476, entitled “Pyranoindazoles and their use for treatment of glaucoma” is extended to a substrate containing a secondary carbamate in the sidechain linked to N-1. The protection of primary amino groups as secondary carbamates is well known in the art. Typical secondary carbamates are a benzyl carbamate NHCO₂CH₂Ph, abbreviated NHCbz, and a t-butyl carbamate NHCO₂t-Bu, abbreviated NHBoc. Greene et al., Protective Groups in Organic Synthesis, 3^{rd} edition, John Wiley and Sons, 1999, pages 518-525 and 531-537.

In one embodiment of the invention of U.S. Pat. No. 6,696,476, the cyclization to form the pyranoindazole is effected by reacting a dihalide with a reducing agent specifically n-butyllithium. The reaction proceeds by metal-halogen exchange to give an arylmetallic intermediate, which displaces the aliphatic halide to generate the pyranoindazole. The application of these general reaction conditions, which can be denoted reductive cyclization or Parham cyclization for pyran ring formation, is discussed by Hodgetts, Tetrahedron Letters, Vol. 41:8655, 2000. One molar equivalent of the organometallic reagent is sufficient to effect the reaction; in practice, slightly more than one molar equivalent of the organometallic reagent can be used to ensure consumption of adventitious moisture. Typically, up to about 1.2 molar equivalents of the organometallic reagent is used.

However, in a case where a dihalide comprises a secondary carbamate, the hydrogen atom bonded to nitrogen (hereafter, the N—H) in the secondary carbamate is sufficiently acidic to consume the organometallic reagent in an acid-base reaction. Therefore the secondary carbamate must be modified in such a way that it does not interfere with the cyclization. In the process of the present invention, a base is deliberately introduced to remove the N—H.

It is advantageous to use a base whose conjugate acid exits the reaction medium, such as via a gas. Such bases include metal hydrides and organometallic reagents. Examples of organometallic reagents are Grignard reagents and organolithium reagents. In certain embodiments of the present invention, the base is preferably chosen from a group of organometallic reagents that possess little or no capability to effect metal-halogen exchange. The reason for this is to avoid premature metal-halogen exchange, which can result in quenching of the arylmetallic intermediate thus formed by an N—H from unreacted starting material. The analogous quenching of an arylmetallic intermediate by an O—H from unreacted starting material is discussed by Gallagher et al., J Am Chem Soc, Vol. 113:7984, 1991. Preferred bases are primary Grignard reagents and aryl Grignard reagents. More preferred are methyl and ethyl Grignard reagents. Optionally, an indicator is used to signal the complete removal of N—H by the base. Indicators suitable for use with Grignard reagents are discussed by Bergbreiter et al., J Org Chem, Vol. 46:219, 1981.

The removal of the N—H by a base also has the advantage of greatly decreasing the susceptibility of the adjacent carbonyl group of the carbamate to undesired reactions. Thus an NHBoc group upon exposure to lithium aluminum hydride does not undergo reduction, because the lithium aluminum hydride functions as a base to remove the N—H: Goel et al., Organic Syntheses, Collective Vol. 8:68, 1993.

Nicolaou et al., Angewandte Chemie International, Edition 37:2717, 1998, describe the use of a methyl Grignard reagent as a base to remove N—H groups of peptidic secondary carboxamides, followed by the use of an isopropyl Grignard reagent to effect metal-halogen exchange to form an arylmetallic intermediate.

Bérubé et al., Organic Letters, Vol. 6:3127, 2004, describe the use of one molar equivalent of an isopropyl Grignard reagent as a base to remove the N—H of an NHBoc group, and a second molar equivalent of an isopropyl Grignard reagent to effect metal-halogen exchange to form an arylmetallic intermediate.

Silylation of a secondary carbamate, that is, replacement of the N—H with N—Si, has been used to protect the secondary carbamate from unwanted reactions with strong bases or organometallic reagents: Roby et al., Tetrahedron Letters, Vol. 38:191, 1997. However, silyl carbamates are hydrolyzed readily by adventitious moisture to regenerate the secondary carbamate, making them potentially difficult to store and transfer.

The secondary carbamate can be cleaved to yield a primary amino group: Greene et al. (cited above). Alternatively, the secondary carbamate can be converted into an alkylamino group or a dialkylamino group as described generally by White et al., Organic Syntheses, Collective Vol. 10:305, 2004.

Certain representative compounds that may be formed by the processes of the present invention comprise those represented by (I) below:

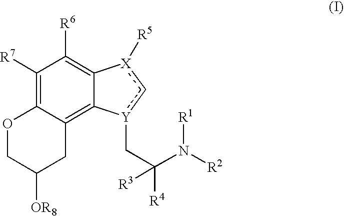

wherein R¹ and R² are independently hydrogen or an alkyl group;

### R³ and R⁴ are independently hydrogen or an alkyl group, or

### R³ and R⁴ and the carbon atom to which they are attached form a cycloalkyl ring;

### R⁵ is hydrogen or a substituted or unsubstituted alkyl group;

### R⁶ and R⁷ are independently hydrogen, alkylthio, or a substituted or unsubstituted alkyl group;

### R⁸ is hydrogen or a substituted or unsubstituted alkyl group;

### X and Y are either N or C, wherein X and Y are different and the dashed bonds denote a suitably appointed single and double bond.

One embodiment of the present invention is a method of making a pyranoindazole comprising reacting with a reducing agent a protected halohydrin comprising a secondary carbamate to form a pyranoindazole. In a preferred embodiment, the protected halohydrin is a bromohydrin silyl ether, such as compound 7 in Scheme 1 below. In other embodiments, various protected halohydrins known to those of skill in the art may be used, such as, for example, a bromohydrin 1-(ethoxy)ethyl ether. A further step may comprise converting the secondary carbamate of the pyranoindazole thus formed via hydrogenolysis or other methods known to the art to form a pyranoindazole having a primary amino group. In a preferred embodiment, the secondary carbamate is a benzyl carbamate.

The reducing agent reacted with the protected halohydrin may be selected from any of a number of reducing agents known to those of skill in the art. In a preferred embodiment, the reducing agent selected is an organometallic. In a more preferred embodiment, the reducing agent is n-butyllithium. In certain embodiments, the reaction of the protected halohydrin with a reducing agent is preceded by reacting the protected halohydrin with a first organometallic compound, preferably a Grignard reagent. In a preferred embodiment, the first organometallic compound is ethylmagnesium chloride.

In another embodiment of the present invention, the compound (R)-1-((S)-2-aminopropyl)-1,7,8,9-tetrahydropyrano[2,3-g]indazol-8-ol can be formed by reacting a bromohydrin silyl ether comprising a benzyl carbamate with a reducing agent to form a pyranoindazole and then cleaving the benzyl carbamate by hydrogenolysis to form (R)-1-((S)-2-aminopropyl)-1,7,8,9-tetrahydropyrano[2,3-g]indazol-8-ol. Other embodiments form bromohydrin silyl ether by forming a bromohydrin from an epoxide and reacting the bromohydrin with a silane to form a bromohydrin silyl ether.

Specific reaction conditions for the above processes can be readily ascertained by those of skill in the art using the information presented above together with conditions provided below in the Examples.

### EXAMPLES

The pyranoindazole (R)-1-((S)-2-aminopropyl)-1,7,8,9-tetrahydropyrano[2,3-g]indazol-8-ol (1) may be synthesized using an embodiment of the present invention according to the following Scheme 1:

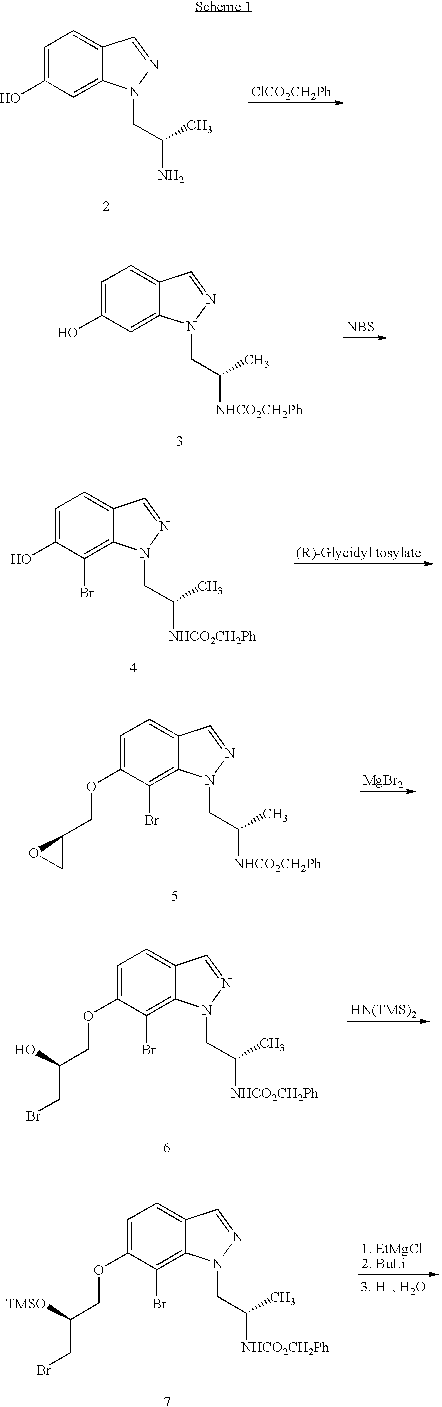

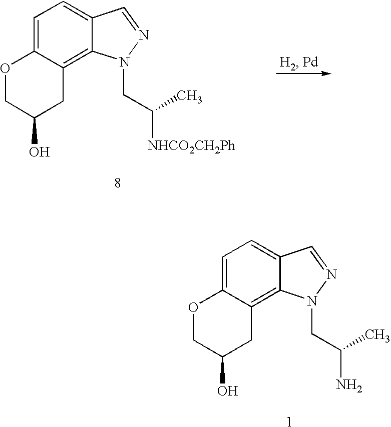

N-Cbz-1-(2(S)-aminopropyl)indazol-6-ol (3). Benzyl chloroformate (29 mL, 0.20 mol) was added to a stirred, ice-cooled solution of 1-(2(S)-aminopropyl)indazol-6-ol (2) (32 g, 0.17 mol) [for preparation of 2 see U.S. Pat. No. 6,998,489, previously incorporated by reference in its entirety; see also WO 04/058725 A1, herein incorporated by reference in its entirety] and triethylamine (23 mL, 0.17 mol) under N₂. The mixture was warmed to ambient temperature and stirred for 16 h. Ammonium hydroxide (200 mL of an aqueous 28% solution) was added to cleave O—Cbz material. After 2 h, the reaction mixture was concentrated to give a solid, which was stirred with 550 mL of 10/1 heptane/ethyl acetate for 16 h and was then collected by filtration and washed with heptane to afford 53 g (98%) of 3.

Bromide 4. N-bromosuccinimide (11 g, 62 mmol) was added in portions over 5 min to a stirred, ice-cooled solution of 3 (20 g, 62 mmol) in 200 mL of tetrahydrofuran under N₂. After 40 min the mixture was concentrated to 40 mL volume. Ethyl ether and ethyl acetate (4:1, 300 mL) were added to dissolve the precipitate. The organic solution was washed with water (2×75 mL), dried (MgSO₄), filtered and concentrated to a slurry. The supernatant was separated and the solid was dried in vacuo to give 4 (19 g, 76%). The supernatant was concentrated and purified by chromatography on silica, eluting with a gradient of CH₂Cl₂/acetone, giving 2.5 g (10%) of 4, for a total yield of 86%.

Epoxide 5. R-(−)-Glycidyl tosylate (13.4 g, 58.8 mmol) and granular K₂CO₃ (14.3 g, 104 mmol) were added to a stirred solution of 4 (19.0 g, 47.0 mmol) in 70 mL of dry DMF. Under N₂, the mixture was heated to 70° C. for 1 h then cooled to RT. EtOAc was added and the mixture was washed 3 times with water, dried (Na₂SO₄), filtered and concentrated. The residue was slurried with Et₂O (75 mL) and the solid was collected and dried to give 19.0 g (88%) of 5.

Bromohydrin 6. MgBr₂ etherate (10.3 g, 40.0 mmol, 1.4 equiv) was added to a stirred, ice-cooled solution of 5 (13.3 g, 28.8 mmol) in 115 mL of dry THF under N₂. After 0.5 h, the ice bath was removed and stirring continued for 2 h. The mixture was poured into 250 mL of 10% aq NH₄Cl and extracted twice with EtOAc. The combined organic extract was dried (MgSO₄), filtered and concentrated. The product was purified by chromatography on silica eluting with 10% acetone/CH₂Cl₂ to give 14.4 g (92%) of 6 as a foam.

Bromohydrin silyl ether 7. Hexamethyldisilazane (6.0 mL, 29 mmol) was added over 15 min to a solution of 6 (14.4 g, 26.6 mmol) and saccharin (50 mg, 1 mol %) in 75 mL of dry MeCN at RT. After a further 30 min, the solution was concentrated and the residue was eluted through a pad of silica with 25 to 33% EtOAc/hexane. Concentration in vacuo afforded 7 (13.8 g, 85%) as an oil.

N-Cbz-Pyranoindazole 8. Ethylmagnesium chloride (2.0M in THF, 16 mL) was added via syringe over 25 min to a stirred, ice-cooled solution of 7 (12.8 g, 20.8 mmol) and 1,10-phenanthroline (0.45 g, 2.5 mmol) in 250 mL of dry THF under Ar, keeping the internal temperature below 5° C., to a persistent purple-pink endpoint. The suspension was cooled in a dry ice/2-propanol bath. n-BuLi (2.5M in hexane, 10 mL, 25 mmol) was added with rapid stirring over 25 min, keeping the internal temperature below −70° C. After a further 10 min the bath was replaced with an ice bath. Stirring at 0 to 5° C. was continued for 30 min. Aqueous 0.5M NaHSO₄ (200 mL) was added causing an exotherm to 20° C. The mixture was stirred at RT for 40 min to effect desilylation. Ethyl acetate (400 mL) was added, the layers were separated and the organic solution was washed with two 200-mL portions of half-saturated brine, dried (MgSO₄), filtered and concentrated, yielding 8.5 g of a yellow foam. Trituration with 50 mL of diethyl ether followed by slurrying with 100 mL of 30% ether-hexane, decantation and drying of the solid in vacuo afforded 7.30 g (92%) of 8.

Pyranoindazole 1. To a suspension of 8 (195 mg, 0.51 mmol) in 5 mL of absolute EtOH under N₂ was added 18 mg of 20% Pd(OH)₂ on C. Hydrogen (1 atm) was introduced and the suspension was stirred for 2.5 h, then filtered and concentrated to give 0.12 g (95%) of 1.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

We claim:

1. A method of making a pyranoindazole comprising:
reacting with a reducing agent a protected halohydrin comprising a secondary carbamate to form a pyranoindazole.

2. The method of claim 1 further comprising the step of:
converting the pyranoindazole via hydrogenolysis to form a pyranoindazole having a primary amino group.

3. The method of claim 1 wherein said reacting is preceded by reacting said protected halohydrin with a first organometallic compound.

4. The method of claim 3 wherein said reducing agent is a second organometallic compound.

5. The method of claim 3 wherein said first organometallic compound is a Grignard reagent.

6. The method of claim 3 wherein said first organometallic compound is methylmagnesium chloride or ethylmagnesium chloride.

7. The method of claim 1 wherein said reducing agent is n-butyllithium.

8. The method of claim 1 wherein said reducing agent is an organometallic compound.

9. The method of claim 1 wherein said secondary carbamate is a benzyl carbamate.

10. A method of making (R)-1-((S)-2-aminopropyl)-1,7,8,9-tetrahydropyrano[2,3-g]indazol-8-ol comprising:
reacting a bromohydrin silyl ether comprising a benzyl carbamate with a reducing agent to form (R)-1-((S)-2-(benzyloxycarbonyl)aminopropyl)-1,7,8,9-tetrahydropyrano[2,3-g]indazol-8-ol;
converting (R)-1-((S)-2-(benzyloxycarbonyl)aminopropyl)-1,7,8,9-tetrahydropyrano[2,3-g]indazol-8-ol via hydrogenolysis to form (R)-1-((S)-2-aminopropyl)-1,7,8,9-tetrahydropyrano[2,3-g]indazol-8-ol.

11. The method of claim 10 wherein said reducing agent is n-butyllithium.

12. The method of claim 10 wherein said reacting is preceded by reacting said bromohydrin silyl ether with a Grignard reagent.

13. The method of claim 12 wherein said Grignard reagent is methylmagnesium chloride or ethylmagnesium chloride.

14. The method of claim 10 wherein said bromohydrin silyl ether is formed by:
forming a bromohydrin from an epoxide;
reacting said bromohydrin with a silane to form said bromohydrin silyl ether.

15. A compound of Formula (I): [chemical structure]
